(19) United States
(12) Patent Application Publication
(10) Pub. No.: US 2009/0169602 A1
(43) Pub. Date: Jul. 2, 2009

(54) Allergy Treatment by Epicutaneous Allergen Administration

(75) Inventors: Gabriela Senti, Zürich (CH); Thomas Kuendig, Bassersdorf (CH)

Correspondence Address: EDWARDS ANGELL PALMER & DODGE LLP, P.O. BOX 55874, BOSTON, MA, 02205, US

(73) Assignee: UNIVERSITÄT ZÜRICH, Zurich (CH)

(21) Appl. No.: 12/085,420

(22) PCT Filed: Nov. 23, 2006
(86) PCT No.: PCT/EP2006/011262

§ 371 Date: Jul. 10, 2008

(30) Foreign Application Priority Data
Nov. 23, 2005 (EP) 05025522.3

(51) Int. Cl. A61K 9/70; A61K 39/35; A61P 37/08

(52) U.S. Cl. 424/448; 424/275.1; 424/192.1

(57) ABSTRACT

The invention provides pharmaceutical compositions, kits, and methods for the treatment of allergy. The compositions are adapted for epicutaneous administration and comprise an allergen and at least one pharmaceutically acceptable excipient. They may be designed as adhesive patches, intradermal delivery devices, ointments, gels, sprays, or similar types of formulation suitable for administration to the skin. Furthermore, the invention provides the use of such compositions in the treatment of allergy. In particular, the compositions are administered to pre-treated skin, wherein the pre-treatment comprises partial or complete dekeratinisation of the epidermis at the selected site of administration.

### BACKGROUND OF THE INVENTION

Over the last century, allergies have become increasingly prevalent in all industrialized nations. Around 100 years ago, hay fever was a rare disease with approximately 1% of the population suffering. Today around 20% of the European population suffer from pollen allergy, and roughly one third of the population has an allergy to pollen, animal dander, house dust mites or food.

The most frequent allergy which is encountered in clinical practice is seasonal rhinoconjunctivitis or hay fever. It is an inflammatory condition of the mucosa that develops when allergen interacts with IgE that is bound to mast cells in sensitized individuals. Characteristic clinical manifestations include nasal itching, sneezing, rhinorrhea, conjunctival redness, and lacrimation. Skin sensitization is predominantly caused by grass pollen (12.7% of the population), followed by house dust mite (8.9%), silver birch pollen (7.9%) and cat epithelia (3.8%) (Wuthrich B. et al., Int Arch Allergy Immunol 1995; 106: 149-56). Allergic rhinitis usually starts in childhood, on average at an age of around 10 years. Its prevalence is increasing, especially in urban areas, for uncertain reasons. Grass pollen is the major cause of pollinosis in many parts of the world. Grass induced pollinosis is also the most common pollen allergy in Europe, although its frequency differs regionally.

Although many assume that allergic rhinitis is more of a nuisance than a clinically significant health problem, it is responsible for $6 billion annually in health care costs in the United States, and frequently causes absence or poor performance at work and school (Durham S. R. et al., N Engl J Med 1999; 341: 468-75).

Current Treatment and Prognosis: Management begins with efforts to prevent exposure to allergens, supplemented by drug therapy. Second generation Hi receptor antagonists and local mast cell stabilizers control symptoms in patients who have mild or moderate rhinitis. Intranasal glucocorticoids must be added for patients with more severe disease. However, these pharmacological treatments are merely symptomatic and have no long lasting beneficial effect on the course of the allergy itself. Allergy to pollen, animal dander or house dust mites usually starts as a mild rhinitis and conjunctivitis, but tends to worsen every year. In about one third of cases the mucosa of the deeper parts of the respiratory tract will become more and more involved, finally leading to asthma. Another third of the patients that initially suffer only from hay fever will develop allergies to foods, as many fruits and vegetables contain proteins that are highly homologous to pollen proteins. The above described course of the allergy with development of asthma and spreading to food allergy cannot be influenced by symptomatic pharmacological treatment, but would require a modulation of the immune system, i.e. allergen specific immunotherapy.

Noon and Freeman reported in 1911 that a majority of patients who had rhinitis had symptomatic improvement after receiving injections of a grass pollen extract (Lancet 1911; 1: 1572-3). Prolonged courses of injections of biologic extracts, popularly known as “allergy shots,” remain the hallmark of therapy for allergic rhinitis. Allergen immunotherapy, also known as desensitization or hyposensitisation, is the practice of administering gradually increasing quantities of an allergen extract to an allergic patient to ameliorate the symptoms associated with the subsequent exposure to the causative allergen. In an ambulatory setting, the allergen dose, for instance standardized grass pollen solution, is increased weekly for 16 weeks. When the maintenance dose is reached, injections are given at 1-month to 2-month intervals (Golden D. B. et al., J Allergy Clin Immunol 1981; 67: 4824). Immunotherapy is the only treatment that may affect the natural course of allergic diseases, and it may also prevent the development of asthma in patients with allergic rhinitis.

However, with conventional subcutaneous desensitization the duration of treatment is around 3-5 years and usually comprises around 30-100 allergen injections. As high allergen doses have to be injected, allergic side effects may occur, and patients must stay under medical supervision for at least 1 hour. Therefore, there is a need for a treatment with lower antigen doses, such that side effects are reduced and the patient would not have to stay under medical supervision or could even treat himself at home. There is furthermore the need for a treatment which is needle-free and painless.

### SUMMARY OF THE INVENTION

In a first aspect, the invention provides a pharmaceutical composition for epicutaneous administration comprising an allergen and at least one excipient. Preferably, the composition is in the form of an adhesive patch, an intradermal delivery device, a liquid, a gel, a spray, or a foam. The antigen may be selected from plant pollen, dust, animal dander, house dust mites, fungal spores, food, or the venom of ants, bees, or wasps. The composition is useful for the treatment of patients suffering from an allergy to the antigen, and in particular for desensitization therapy.

In a second aspect, the invention provides a pharmaceutical kit which comprises such pharmaceutical composition, and which kit further includes printed instructions to perform a pre-treatment of a selected area of the skin and to subsequently administer the composition to the pre-treated area of the skin. Preferably, the kit further comprises a means to pre-treat the selected area of the skin, such as an adhesive tape.

In a further aspect, the invention provides the use of a pharmaceutical composition comprising an antigen and at least one excipient, which composition is adapted for epicutaneous administration, for the treatment of allergy. Moreover, the invention provides the use of a combination of such pharmaceutical composition and a means for pre-treating a selected area of the skin for the treatment of allergy.

In a yet further aspect, the invention provides a method of treating a patient suffering from an allergy to an antigen. The method comprises the steps of (a) pre-treating a selected area of the skin, and subsequently (b) administering a pharmaceutical composition to the pre-treated area of the skin. The pharmaceutical composition comprises an antigen and at least one excipient. Preferably, the antigen is selected from the group consisting of natural allergens, modified natural allergens, synthetic allergens, recombinant allergens, allergoids, and combinations thereof.

Further aspects of the invention will become obvious on the basis of the following detailed description of the invention, the examples, and the patent claims.

### BRIEF DESCRIPTION OF THE DRAWINGS

FIG. 1 illustrates the cross-section of an example of an adhesive patch having a matrix-type design.

FIG. 2 illustrates the cross-section of an example of an adhesive patch representing a reservoir system.

FIG. 3 illustrates the effect of 12 adhesive patches administered to each of 20 individuals at weekly intervals on the severity of allergy symptoms (NPT scores).

### DETAILED DESCRIPTION OF THE INVENTION

According to a first principal aspect of the invention, a pharmaceutical composition for epicutaneous administration is provided, which composition comprises an allergen and at least one excipient.

A pharmaceutical composition, as used herein, is a composition comprising at least one bioactive agent, which is adapted to be administered to a human or another animal, and which is useful for the maintenance of a state of health or the prevention, control, alleviation or treatment of symptoms, conditions, or diseases. Epicutaneous administration is understood as a mode of administration in which a composition is placed onto the skin, and wherein the composition releases a bioactive agent onto the skin, into the skin, or through the skin to other tissues of the organism. Often, epicutaneous administration is conducted on intact skin; however, it may also be conducted on impaired skin. Epicutaneous administration may also be referred to as topical, dermal, or intradermal administration, and if it involves the potential permeation of the skin by the bioactive agent, it may be referred to as transcutaneous, percutaneous or transdermal administration. Unless stated otherwise, epicutaneous administration is used within the context of the present invention for any type of administration to the skin, whether involving transdermal delivery or not. As used herein, the expression “for epicutaneous administration” is understood as meaning the same as being adapted for epicutaneous administration. In other words, the composition of the invention is composed and processed in such a way that it is suitable for epicutaneous administration by generally accepted criteria.

An excipient is any pharmacologically inert, pharmaceutically acceptable substance or mixture of substances useful for formulating a pharmaceutical composition. Examples of potentially useful excipients include solvents, cosolvents, diluents, surfactants, co-surfactants, thickeners, film-forming agents, stabilisers, antioxidants, solubilisers, pH-adjusting agents, colouring agents, and the like.

An allergen is any compound, substance, or material which is capable of evoking an allergic reaction. Allergens are usually understood as a subcategory of antigens, which are compounds, substances, or materials capable of evoking any immune response. For carrying out the invention, the allergen may be selected, inter alia, from natural or native allergens, modified natural allergens, synthetic allergens, recombinant allergens, allergoids, and mixtures or combinations thereof. Of particular interest are allergens which are capable of causing an IgE-mediated immediate type hypersensitivity.

Examples of preferred allergens include compounds or compound mixtures representing—or being obtained from—plant pollen, dust, animal dander, house dust mites, fungal spores, food, or the venom of ants, bees, or wasps. The allergens or allergen mixtures may be used in their native form, or after physical or chemical modification or derivatisation. Optionally, recombinant or synthetic analogues of the a.m. allergens may be incorporated into the composition.

In terms of their chemical or biochemical nature, the preferred allergens may represent native or recombinant proteins or peptides, fragments or truncated versions of native or recombinant proteins or peptides, fusion proteins, synthetic compounds (chemical allergens), synthetic compounds which mimic an allergen, or chemically or physically altered allergens, such as allergens modified by heat denaturation.

Among the particularly preferred allergens are plant pollen or allergenic components of plant pollen, such as pollen from grasses, trees, and weeds which are capable of causing hay fever, or any modifications or analogues as outlined above. Examples of grass pollen allergens include pollen allergens from maize, Timothy grass, meadow grass, Bermuda grass, bluegrass, brome, paspalum, orchard grass, perennial rye, sweet vernal, meadow fescue, velvet, wild oat, perennial rye, common reed, June (Kentucky blue), red top, Johnson, cultivated rye, cultivated oat, cultivated wheat, meadow foxtail, Bahia, wild rye, Canary grass, couch, Sudan grass, salt grass, and any mixture thereof.

Examples of weed pollen allergens are allergens from common ragweed, Western ragweed, giant ragweed, false ragweed, wormwood, ox-eye daisy, Russian thistle, golden rod, mugwort, pellitory, nettles, plantain, duck weed, fat hen, sorrel, pigweed, goosefoot, dandelion, goldenrod, helianthus, sage, cocklebur, clover, alfalfa, rabbitbush, careless weed, saltbush, poverty weed, rough pigweed, yellow dock, dog fennel, and any mixtures of these.

Trees from which pollen allergens may be obtained to carry out the invention include, for example, alder, elm, olive, ash, hazel, pine, beech, heath, plane, birch, hickory, poplar, chestnut, hornbeam, lime, linden, maple, ti, cypress, myrtle, wattle, Japanese cedar, mulberry, walnut, Western red cedar, oak, willow, and any mixtures of these. Optionally, the allergens may represent mixtures of grass-, weed-, and/or tree pollen allergens.

Another group of preferred allergens are allergens from fungi, in particular from mold spores, such as from Penicillium n., Cladosporium h., Aspergillus f., Mucor r., Candida a., Alternaria a., Botrytis c., Helminthosporium h., Fusarium m., Fusarium s., Stemphylium b., Rhizopus n., Aureobasidium p. (Pullularia), Phoma b., Epicoccum p., Trichoderma v., Curvularia s., Trichophyton m., Grass Smut, Malassezia pachydermatis, Cephalothecium, Hormodendrum, Mucor, Rhisopus, or any mixtures thereof.

A further group of preferred allergens are based on animal venoms, such as venoms from the honeybee, yellow jacket, wasp, paper wasp, yellow hornet, cockroach, flea, deer fly, black ant, housefly, red ant, mosquito, fire ant, moth, horse fly, or any mixtures thereof.

A further group of preferred allergens are based on other animal products which are not venoms, in particular animal hair, animal dander, excretions of (house) dust mites, calyx components of cockroaches, etc.

Natural allergens which are not airborne, but typically encountered in food, and which are also preferred allergens in the context of the present invention, include, for example, allergens from peanuts, nuts, sesame, seafood, milk, egg, peas, beans, soy beans, other legumes, wheat, maize, or any mixtures thereof.

In one of the particularly preferred embodiments, the allergen is a mixture of optionally modified grass, weed, and/or tree allergens, which may be adsorbed onto an aluminium or calcium salt.

Allergens obtained through recombinant means or peptide synthesis, as well as antigens natural sources or extracts, may be purified by means of the antigen's physical and chemical characteristics, preferably by fractionation or chromatography.

The allergens may also be small molecules mimicking the IgG or IgE binding sites of the original allergen. A group of allergens or allergoids which may be used in the composition of the invention is represented by peptides or peptide fragments (e.g. T cell peptides) which are derived from biological allergens and designed to retain the immunomodulatory effects of the parent allergen through direct activity on T cells, but which lack, or show reduced, IgE-mediated systemic effects. Typically, such peptides comprise from about 8 to about 20 amino acid residues. Optionally, they are cyclic, and/or their C-terminus may be amidated, and/or their N-terminus may be acetylated or otherwise acylated. Non-limiting examples of such potentially suitable peptides have the amino acid sequences, EICPAVKRDVDLFLTGT, FLTGTPDEYVEQVAQY, EQVAQYKALPVVLENA, KALPVVLENARILKNCV, RILKNCVDAKMTEEDKE, KMTEEDKENALSLLDK, KENALSVLDKIYTSPL, LTKVNATEPERTAMKK, TAMKKIQDCYVENGLI, SRVLDGLVMTTISSSK, ISSSKDCMGEAVQNTV, AVQNTVEDLKLNTLGR. Further guidance on the selection and optional features of such peptides may be derived from the literature, e.g. from C. Alexander et al., The effect of Feld 1-derived T-cell peptides on upper and lower airway outcome measurements in cat-allergic subjects, Allergy 2005: 60: 12691274.

In one of the particularly preferred embodiments, the composition of the invention is characterised in that it is free of an immunological adjuvant. In a further embodiment, the composition does comprise one or more adjuvants.

Immunological adjuvants may be defined as substances used in combination with a specific antigen that produce a more robust immune response than the antigen alone. This broad definition encompasses a very wide range of materials. Some immunological adjuvants that are used in many marketed vaccine products include mineral salts, in particular calcium phosphate, aluminium phosphate, and aluminium hydroxide. More effective immunostimulatory adjuvants include immunostimulatory oligodeoxynucleotides, immunostimulatory RNA; proteins, including antibodies, or smaller synthetic chemical entities that bind to immunostimulatory or co-stimulatory receptors in general, such as Toll-like receptors. In particular, a suitable adjuvant may be selected from the group including saponins (such as QS21), cytokines (such as IL-2, IL-12), MDP derivatives, LPS, MLP and its derivatives, GM-CSF, lipopeptides, and imiquimod. Another group of potentially suitable adjuvants is represented by colloidal particles, such as lipid particles, e.g. liposomes, virosomes, Iscoms, cochleates; or polymeric nano- or microparticles, e.g. poloxamer or polylactide-co-glycolide particles.

The amount of allergen in the composition of the invention should be selected in consideration of the nature of the selected allergen. In most cases in which a native or modified biological allergen or mixture of allergens is used, the allergen content should be in the range from 0.001 to 1,000 μg, preferably from 0.01 to 100 μg, or from about 0.1 to about 10 μg, or from about 1 to about 10 μg. In the case that a mixture of allergens is used, the total amount of allergens may be somewhat higher, such as in the range from about 0.1 to about 10,000 μg, from about 1 to about 1,000 μg, from about 5 to about 500 μg, from about 10 to about 100 μg, or from about 20 to about 50 μg.

However, it should be taken into consideration that the immunological potency may vary substantially between the various types of allergens and allergoids (including modified variants and derivatives, etc.) mentioned above. Therefore, it may be useful to use alternative parameters rather than mass to describe the strength of the allergen(s) incorporated in the composition of the invention. For example, Bioequivalent Allergy Units (BAU) or the Reactivity Index (R.I.) may be used to quantify the immunogenic activity. In terms of BAU, a suitable allergen content in the composition is preferably selected within the range from about 0.001 to about 10 BAU.

The composition may be formulated as an adhesive patch, an intradermal delivery device, a liquid, a gel, a spray, a foam, or any other type of dosage form that is suitable for epicutaneous administration. Depending on the dosage form design, the at least one excipient is selected to provide a formulation which is appropriate for administration to humans, and which is effective and safe.

As used herein, an adhesive patch is a flat, tape-like dosage form adapted for being placed on the skin. In the context of drug delivery, adhesive patches may also be referred to as transdermal patches, transdermal delivery systems, transdermal therapeutic system, skin patches and the like.

Various optional designs may be selected to make an adhesive patch comprising an allergen according to the invention. In one of the embodiments, the patch is designed as a drug-in-adhesive or matrix-type system, which means that the allergen (which represents the bioactive ingredient or “drug”) is incorporated within a pressure-sensitive adhesive layer of the patch. In another embodiment, the patch may be designed as a reservoir system, meaning that the bioactive agent is incorporated within a non-adhesive, typically liquid or semisolid reservoir from which it is released to the skin through a permeable membrane layer. According to a yet further principal design, the patch may be formulated as a hydrogel system.

In the case that a drug-in-adhesive or matrix design is chosen, useful optional features of the patch may be selected according to formulation techniques which are per se known. In particular, it is preferred that the patch further comprises a backing layer which is substantially impermeable to the allergen incorporated in the adhesive layer, and/or a peelable protective layer, also referred to as release liner, to protect the patch during storage and handling until it is administered.

FIG. 1 shows a schematic cross-sectional drawing of one example of a matrix-type adhesive patch. The adhesive patch (1) comprises a backing layer (2) and a pressure-sensitive adhesive layer (3) in which the allergen is incorporated. A removable release liner (4) protects in particular the adhesive layer during storage and handling. Prior to administration, it is removed. Due to the many options with regard to the materials which may be used for composing the various layers, the thicknesses of the layers relative to each other in the drawing are merely illustrative. Furthermore, all layers are represented in the drawing with an enlarged thickness.

The adhesive layer which also forms the matrix for the bioactive agent may be formulated on the basis of any of the common pressure-sensitive adhesives used for transdermal systems. Examples of pressure-sensitive adhesives include polyacrylates, polysiloxanes, polyisobutylene, polyisoprene, polybutadiene, styrenic block polymers, and the like. Examples of styrenic block copolymer-based adhesives include, for example, styrene-isoprene-styrene block copolymer, styrene-butadiene-styrene copolymer, styrene-ethylenebutene-styrene copolymers, and di-block analogs thereof. All of these materials are understood as excipients according to the invention.

The acrylate polymers that are considered useful in the context of the invention are preferably comprised of a copolymer or terpolymer comprising at least two or more exemplary components selected from the group comprising acrylic acids, alkyl acrylates, methacrylates, copolymerisable secondary monomers or monomers with functional groups. Examples of appropriate monomers include acrylic acid, methacrylic acid, ethyl acrylate, butyl acrylate, methoxyethyl acrylate, acrylamide, butyl methacrylate, hexyl acrylate, hexyl methacrylate, 2-ethylbutyl acrylate, hydroxypropyl acrylate, 2-ethylbutyl methacrylate, isooctyl acrylate, isooctyl methacrylate, 2-ethylhexyl acrylate, methoxyethyl methacrylate, 2-ethylhexyl methacrylate, methoxyethyl acrylate, decyl acrylate, decyl methacrylate, dodecyl acrylate, dodecyl methacrylate, tridecyl acrylate, tridecyl methacrylate, hydroxyethyl acrylate, dimethylacrylamide, acrylonitrile, dimethylaminoethyl acrylate, dimethylaminoethyl methacrylate, tertbutylaminoethyl acrylate, tert-butylaminoethyl methacrylate, and the like.

Polyisobutylenes are elastomeric polymers commonly used in pressure sensitive adhesives. Their molecular structure lends itself to chemical stability and high resistance to aging. Polyisobutylenes can be used as primary base polymers and/or as tackifiers in the adhesive layers of patches. As primary-base polymers, they exhibit relatively weak adhesion to the skin and will often need to have tackifiers added to the adhesive formulation. Such tackifiers can be polyisobutylenes of lower molecular weight, or rosin ester resins.

Specific examples of silicone polymers, or polysiloxanes, include for example silicone pressure sensitive adhesives which are a based on two major components: a polymer, or gum, and a tackifying resin. The polysiloxane adhesive may be prepared by cross-linking the gum, typically a high molecular weight polydiorganosiloxane with the resin to produce a three-dimensional silicate structure via a condensation reaction in an appropriate organic solvent.

Moreover, the adhesive layer may optionally comprise one or more additional excipients or components such as permeation enhancers, plasticisers, additives, stabilizers, dyes, diluents, tackifying agent, pigments, carriers, inert fillers, antioxidants, excipients, gelling agents, anti-irritants, and other excipients generally known to be useful in formulating patches. According to one of the preferred embodiments, the composition of the adhesive layer is substantially hypoallergenic.

The backing layer may be an occlusive or non-occlusive material comprising a woven fabric, polyvinylidene chloride, polyethylene, low density polyethylene, medium density polyethylene, high density polyethylene, polyester, ethylene vinyl acetate, polyethylene terephthalate, polyvinyl acetate, polybutylene terephthalate, polyurethane, coated paper, aluminium and the like, or a combination of any of these. Structurally, the backing layer may be monolithic or laminated material. A suitable thickness for the backing layer is preferably from about 5 to about 500 μm, depending on the material that it is composed from. If it is made predominantly from polymeric materials, the thickness of the backing layer should preferably not exceed about 250 μm, and more preferably not exceed about 150 μm. In one of the preferred embodiments, the composition of the backing layer and its thickness are selected to render it substantially permeable.

A release liner may generally consist of the same types of materials as a backing layer, except that its surface which is to be in contact with the adhesive layer should be siliconised in order to enable its easy detachment for administration. A release liner may also be somewhat thicker and less flexible than the backing layer.

Further guidance on useful optional features of a matrix patch may be derived e.g. from U.S. Pat. Nos. 4,588,580, 5,985,317, 5,783,208, 5,626,866, 5,227,169, whose disclosure is incorporated herein by reference.

According to another embodiment, the composition of the invention is formulated as a reservoir patch. Preferably, the allergen is incorporated within a liquid or semisolid excipient or mixture of excipients which forms the reservoir. The reservoir is positioned between a backing layer and a porous or otherwise permeable membrane which ensures the physical integrity of the patch system and which may also contribute to the control of the release of the incorporated bioactive ingredient. Thus, the membrane forms an important component of the patch in this embodiment.

Those skilled in the art will recognise a variety of materials which may be used as the membrane. For example, the membrane may be a dense or homogeneous membrane made of a material that is inherently permeable to the allergen and other components of the reservoir, which are to be conveyed to the membrane. Alternatively, it may be made of a microporous material whose pores are filled with a material that can be permeated by the allergen. Further, the membrane may be a layer made of a specific polymeric material. For example, it may include an amount of ethylene vinyl acetate copolymer. In the case of dense membranes, the active compound molecules travelling from the storage layer to the membrane typically dissolve in the membrane material and diffuse through it. In the case of microporous materials, the active ingredient diffuses through the pores to the membrane. Examples of materials for making dense membranes are given in U.S. Pat. Nos. 3,598,122 and 4,650,484. Examples of materials for making microporous membranes are provided in U.S. Pat. Nos. 3,797,494 and 4,031,894.

The reservoir comprises the allergen dispersed or dissolved in a liquid or semisolid carrier. Appropriate liquid carriers may be based on physiologically acceptable liquid solvents such as water, ethanol, acetone, propanol, isopropanol, mineral oil, silicone, polyethylene glycol, polypropylene glycol, liquid sugars, waxes, petroleum, or glycerol, optionally in combination with a cosolvent. The viscosity of the liquid carrier may be increased by the incorporation of a thickener or gelling agent. Optionally, the reservoir composition may be formulated in analogy to common formulation principles for dermal solutions, suspensions, emulsions, gels, ointments, creams, pastes, foams, sprays and the like. If the viscosity is high enough to render the reservoir semisolid, it may even be possible to design the patch without a release controlling membrane. Solvents, cosolvents, thickeners, gelling agents and any other pharmacologically inert materials useful for making the reservoir are understood as excipients according to the invention.

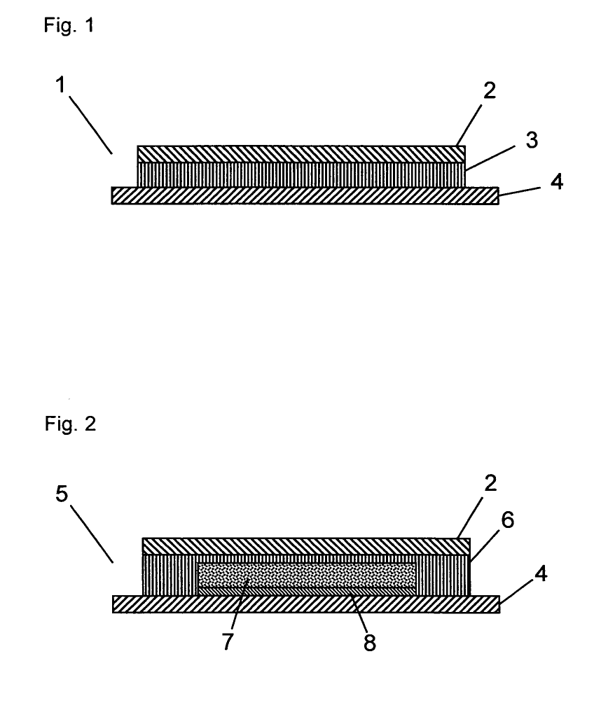

FIG. 2 shows a schematic cross-sectional drawing of one example of an adhesive patch representing a reservoir system. The adhesive patch (5) comprises a backing layer (2) and a pressure-sensitive adhesive layer (6). A removable release liner (4) protects in particular the adhesive layer during storage and handling. Prior to administration, it is removed. A semisolid or liquid reservoir (7) is positioned between the adhesive layer (6) and the release liner (4) in such a way that the adhesive layer (6), which has larger planar dimensions than the reservoir (7), extends over the latter's planar dimensions. The patch (5) further comprises a membrane (8) which is permeable to the allergen. Due to the many options with regard to the materials which may be used for composing the various layers, the thicknesses of the layers relative to each other in the drawing is merely illustrative. Furthermore, all layers are represented in the drawing with an enlarged thickness.

In another embodiment, the patch design is that of a hydrogel patch. Hydrogels are mixtures of water and a gelling agent, such as a hydrophilic polymer. In general, hydrogels form a three-dimensional lattice of polymer chains that retains an aqueous solution in a flexible, stable shape. Preferred hydrogels contain gelling agents distributed substantially uniformly throughout the carrier liquid, which is typically aqueous and may contain an alcohol and/or an oil.

In the case of a reservoir-type patch or a hydrogel-type patch, the reservoir or the hydrogel itself may not always be rendered sticky enough to provide sufficient adhesion to the skin. In these cases, it is preferred that an additional adhesive layer without active ingredient is used, such as in form of an adhesive ring around the hydrogel or the liquid reservoir, to ensure skin adhesion; or that the planar dimensions of the adhesive layer are larger than those of the reservoir so that there is a ring-shaped area of the adhesive layer around the reservoir or hydrogel. According to another preferred embodiment, the composition of the adhesive material is substantially hypoallergenic.

For the selection of appropriate backing layers and release liners for reservoir- and hydrogel-type patches, the same general principles apply as have been mentioned above in the context of matrix-type patches.

Regardless of whether the patch is designed as a matrix-type, reservoir-type, or hydrogel-type patch, the shape of the patch may be selected to represent a square, a rectangle, a circle, an ellipse, an ellipsoid, or have an irregular shape. The area of the patch which is in contact with the skin is preferably selected in the region from about 1 to about 400 cm², and more preferably from about 2 to about 200 cm². In further preferred embodiments, the skin contacting area of the patch is in the range from about 4 to about 100 cm², from about 5 to about 80 cm², from about 10 to about 50 cm², or about 15 cm², respectively.

Particularly in the case of a reservoir- or hydrogel-type patch, but potentially also in the case of a matrix patch, the patch may exhibit a release area which is adapted to release the allergen into the skin, which release area is smaller than the total skin contacting area of the patch. This is the case, for example, in those embodiments in which the liquid reservoir, the hydrogel, or the allergen-containing matrix layer is not sufficiently adhesive to ensure adherence to the skin, and in which the proximal side of the patch (i.e. that which is in contact with the skin) exhibits an area around the release area which is more adhesive but substantially allergen-free. In a related embodiment, at least a portion of the release area is not covered (at the proximal side) with the pressure-sensitive adhesive layer.

In these cases, it is preferred that the total skin contacting area of the patch does not exceed the release area by more than about 200%. More preferably, the total skin contacting area of the patch exceeds the release area by less than about 100%, or less than about 75%, or even less than about 50%, respectively. The release area itself has preferably dimensions in the range from about 0.5 to about 200 cm², or more preferably from about 0.5 to about 50 cm², or from about 1 to about 25 cm².

According to a further embodiment, the composition of the invention is designed as an intradermal delivery device rather than an adhesive patch. While the adhesive patches disclosed above are passive delivery systems, which means that the allergen is released from the composition and is taken up by the skin without the application of thermal, mechanical, electrical, ultrasonic or magnetic energy, intradermal delivery devices are usually active delivery systems which do make use of such energy.

For example, methods and delivery devices using electrical pulses to transport active molecules into or through the skin are per se known from U.S. Pat. No. 5,019,034, U.S. Pat. No. 5,387,189, U.S. Pat. No. 6,148,232 and U.S. Pat. No. 5,318,514, which are incorporated herein by reference. The methods are also referred to as skin electroporation.

In another embodiment, heat ablation is used for the intradermal delivery of the antigen, such as disclosed in U.S. Pat. No. 5,885,211, which is incorporated herein. In yet another embodiment, delivery devices incorporating microneedles may be used, such as disclosed in U.S. Pat. No. 6,334,856, which is also incorporated herein. Optionally, ultrasound may be used in combination with an electrical field to provide for the delivery of the allergen into the skin, such as described in U.S. Pat. No. 6,041,253, which is also incorporated herein.

In another embodiment, the composition of the invention is formulated as a liquid, a gel, a spray, or a foam. As used herein, a liquid formulation is characterised by the liquid state of at least the coherent (or continuous) phase of the composition. If the liquid is a liquid solution, it comprises only one phase which is at the same time coherent. Alternatively, a liquid formulation may also incorporate one or more further phases which are dispersed in the continuous phase and which may or may not be liquid. For example, a suspension is a liquid comprising a dispersed solid phase, and an emulsion is a liquid comprising a dispersed liquid phase.

Excipients and formulation techniques for liquid compositions for administration onto the skin are generally known to the skilled person. Among the preferred liquid constituents for such compositions are water, ethanol, and isopropanol. Optionally, one or more organic co-solvents may also be incorporated. Depending on its chemical nature, the allergen may be dissolved, colloidally dispersed or suspended in the liquid phase. Preferably, the allergen is incorporated in a dissolved or colloidally dispersed state.

Further optional excipients for formulating the liquid composition include pharmaceutically acceptable thickeners, gelling agents, surfactants, co-surfactants, stabilisers, colouring agents, pH-adjusting agents such as acids, bases, and buffer salts, lipids, oils, preservatives, sugars, sugar alcohols, bioadhesive agents, film-forming polymers, plasticisers, and permeation enhancers. In one of the preferred embodiments, the liquid composition comprises at least one film-forming polymer such as a methacrylate copolymer, optionally a plasticiser to adjust the mechanical properties of the film-forming polymer. Due to the incorporation of the film-forming excipient, the composition will, after administration and evaporation or absorption of the liquid constituents, form a thin, flexible film on the skin from which the allergen is continuously released into the skin. If the viscosity of the composition is sufficiently low, it may advantageously be presented and administered in the form of a spray.

In another embodiment, the composition of the invention is formulated as a gel. As used herein, a gel is a semisolid material having viscoelastic properties. It behaves like an elastic solid material upon the exertion of low mechanical shear stress, but like a viscous liquid under high shear stress. The yield point, or yield stress, defines the threshold at which the gel begins to deform plastically.

Excipients and formulation techniques for gel compositions for administration onto the skin are generally known to the skilled person. The selection of excipients also depends on which type of gel formulation is chosen. Optional types of gels include, for example, clear monophasic gels, such as viscoelastic hydrogels based on a chemically or physically crosslinked hydrocolloid and water; lipophilic gels or ointments, such as vaseline-based jellies; and semisolid emulsions, such as o/w- or w/o-creams comprising a hydrophilic (usually aqueous) and a lipophilic (or oily) phase.

Furthermore, formulations may be used as are known for testing purposes for allergy, i.e. allergy testing patches or corresponding test devices applying allergens to the skin for studying the reaction of the patient's skin and determining whether a particular patient is allergic to a particular allergen.

In a further principal aspect of the invention, a method of treating a patient suffering from an allergy to an antigen is provided. The method comprises the steps of (a) pre-treating a selected area of the skin, and subsequently (b) administering a pharmaceutical composition to the pre-treated area of the skin. The pharmaceutical composition is a composition having the same features as discussed above. In particular, it comprises an antigen and at least one excipient, wherein the antigen is selected from the group consisting of natural allergens, modified natural allergens, synthetic allergens, recombinant allergens, allergoids, and combinations thereof.

The pre-treatment of the area of the skin which is also selected for the administration of the composition is preferably conducted in such a way that the skin is irritated and/or that the keratinised epithelial layer (stratum corneum) of the epidermis is at least partially disrupted or removed. For example, skin pre-treatment may comprise rubbing, administration of an organic solvent, administration of a keratinolytic agent, depilation, abrasion, ablation, electroporation, microporation, tape-stripping, or a combination of any of these.

In one of the preferred embodiments, the pre-treatment is conducted such that the epidermis of the selected skin area is at least partially dekeratinised, i.e. the keratinised layer is removed completely or in part. Particularly preferred is the application of tape-stripping for partial or complete dekeratinisation. Alternatively, the keratinised layer may be removed by the use of other mechanical means, such as a razor blade or another device having a blade, or abrasive paper (sandpaper).

As used herein, tape-stripping is understood as the application and subsequent removal of an adhesive material to the skin, whereby at least some of the keratin of the stratum corneum is removed. Depending on the-type of adhesive material, a series of two or more tape-strippings must be performed in order to remove a substantial part, or all, of the keratinised layer. Examples of adhesive materials which may be used for skin pre-treatment include adhesive tapes and waxes, such as facial strip wax sheets. In a preferred embodiment, the pre-treatment is conducted in the form of at least 2, and more preferably at least 3 tape-strippings using an adhesive tape.

Without wishing to be bound by theory, it is believed that the pre-treatment, in particular in the case of tape-stripping, has at least two key effects which contribute to the effectiveness of the method of the invention. Firstly, the at least partial disruption of the keratinised layer which forms the most relevant barrier of the skin to the invasion of an externally administered allergen, effects an increase of the dermal or transdermal permeation of the allergen. Consequently, the allergen molecules have easier access to the Langerhans cells which reside in the basal layer of the epidermis. The Langerhans cells play a crucial role in the immune response to allergens invading the body through the skin as they present such allergens to the T lymphocytes residing in the regional lymph nodes.

Secondly, it is believed that the pre-treatment in itself has an immunostimulatory effect. The partial or complete disruption of the skin barrier function represents an irritation which activates both Langerhans cells and keratinocytes to re-establish the epidermal functions. More specifically, it is believed that the disruption may Initiate a chain of molecular events that leads to the secretion of proinflammatory cytokines such as tumor necrosis factor alpha, interleukin-1, and granulocyte-macrophage colony-stimulating factor by the keratinocytes. The secretion of tumor necrosis factor and IL-1 in particular promote the migration of Langerhans cells from the epidermis to regional lymph nodes.

In further preferred embodiments, the method of the invention is carried out using one or more of the optional features of the pharmaceutical composition as described above.

In another preferred embodiment, the method is repeatedly conducted, i.e. both the pre-treatment step and the administration of the composition comprising the allergen. The regimen according to which the regular or irregular administration interval is selected should take into account the severity of the disease or condition of the particular patient, the immunological sensitivity of the patient to the allergen(s) comprised in the composition, the phase of the therapeutic intervention, the type of formulation etc. Typically, the pre-treatment and administration of the composition is conducted at a frequency ranging from about once a day to about once a year. Preferred regimen include, inter alia, administration every other day, three times per week, twice per week, once a week, and once every two weeks.

The administration may optionally be repeated between one and one hundred times for one course of therapy, and more preferably such course of therapy comprises from 5 to about 20 consecutive applications. Typically, such course of therapy will last for a period of time in the range from about one week to about 6 months, or more preferably from about 2 weeks to about 5 months, for example about 1 month, about 6 weeks, about 2 months, or about 3 months.

The treatment course may begin before, after or during the time of symptom-causing allergen exposure in the environment, such as before, after, or during the pollen season for patients with pollen allergy, or before, after, or during the fall and winter season for patients with dust mite allergy.

Within a course of treatment, the administered dose or potency of the allergen(s) per episode may remain substantially constant, or it may be adjusted, in particular increased over time. If the composition is in the form of an adhesive patch, the dose may be increased by either administering patches loaded with increasing amounts of allergen, or by administering an increasing number of patches per episode of pre-treatment and administration.

For sustaining the positive effects of a therapy that has been conducted for a period of several weeks or months according to one of these regimen, it may also be useful thereafter to continue with a decreased frequency of administration, such as about once every month, about once every 2 months, about once every 3 months, about once every 6 months, or about once a year.

In the case that the composition is formulated as an adhesive patch, the wearing time for a patch should be selected in the range from about 1 minute to about 14 days, and more preferably from about 1 hour to about 7 days. In further preferred embodiments, the wearing time is from about 4 hours to about 2 days, or from about 5 to about 9 hours (such as overnight), or approximately 1 day, or approximately 2 days.

The composition should preferably be administered to the pre-treated skin within about 6 hours after the pre-treatment. More preferably, the administration should be conducted within about 4 hours, 2 hours, 1 hours, 30 minutes, and in particular within less than about 10 minutes after the pre-treatment.

According to a further preferred embodiment, the area of the skin selected for pre-treatment and administration of the composition of the invention is alternated for each consecutive administration. More preferably, a selected skin area which has been used for a first episode of pre-treatment and administration is not used again for a subsequent pre-treatment and administration within a selected period of at least about 3 days after the first pre-treatment. In another embodiment, such selected period is at least about one week, about 10 days, about two weeks, about 3 weeks, or about one month, respectively.

The site of the body where the area of the skin is selected for pre-treatment and administration according to the method of the present invention is preferably one which is only moderately hairy and not excessively affected by sweating. This is particularly contemplated for those embodiments in which the composition of the invention is in the form of an adhesive patch. Examples of potentially suitable sites are the arms, in particular the upper arms, the chest, the abdomen, with some limitation also the thighs and the hips.

In a further aspect, the invention provides the use of a pharmaceutical composition as described above for the manufacture of a medicament for the treatment of allergy. Moreover, it provides the use of a combination of a means for pre-treating a selected area of the skin and a pharmaceutical composition as defined herein for the manufacture of a medicament for the treatment of allergy. According to a particularly preferred embodiment of this use, the means for pre-treating a selected area of the skin is a means for tape-stripping, in particular an adhesive tape, and the pharmaceutical composition is provided in the form of an adhesive patch. Further optional features as have been discussed above in the context of the pharmaceutical composition and the method of treatment also apply by analogy to the use provided herein.

In a further principal aspect of the invention, a pharmaceutical kit is provided, which kit comprises the composition disclosed herein above along with printed instructions to perform a pre-treatment of a selected area of the skin and to subsequently administer the composition to the pre-treated area of the skin. In a preferred embodiment, the pre-treatment specified in the instructions possesses the same features as previously discussed in the context of the method of treatment. In particular, it is preferred that the pre-treatment comprises partial or complete dekeratinisation of the selected skin area, which dekeratinisation is preferably performed as tape-stripping. The instructions may also comprise any of the optional or preferred features of the method of treatment described above.

Advantageously, the kit may comprise not only the composition and the instructions, but also the means for performing the pre-treatment of the skin. This means may be provided in the form of an adhesive tape or a stripping wax. In a further embodiment, a pharmaceutical kit is provided which comprises (a) a pharmaceutical composition in the form of an adhesive patch comprising at least one allergen suitable for hyposensitisation therapy, (b) at least one piece of an adhesive tape which is suitable and adapted for pre-treating a selected area of the skin of a human by tape-stripping, and (c) printed instructions to perform the tape-stripping pre-treatment of the selected area of the skin and to subsequently administer the patch to the pre-treated area of the skin. Optionally, the kit may contain more than one patches. Further optional features as have been discussed above in the context of the pharmaceutical composition and the method of treatment also apply by analogy to the kit provided herein.

The pharmaceutical composition and the kit are, according to the invention, used for the treatment of patients suffering from an allergy. As used herein, an allergy is understood as including any form of latent or manifest type-1 hypersensitivity, also referred to as atopic or IgE-mediated hypersensitivity. This class of hypersensitivity is typically characterised by an excessive activation of mast cells and basophils through immunoglobulin E (IgE) which may result in a systemic inflammatory response that can cause a broad range of mild, moderate, and severe symptoms ranging from a runny nose to a life-threatening anaphylactic shock.

It is believed that the crucial difference between a type I hypersensitivity reaction against an allergen and a healthy humoral response against a foreign body is that, in hypersensitivity, plasma cells predominantly secrete immunoglobulins of type E rather than type M, which normally act against novel antigens, or type G, which are secreted against recognised antigens. IgE binds to Fc receptors on the surface of mast cells and basophils, which are both involved in the acute inflammatory response. Upon its secretion, an IgE binds to the Fc receptors on a mast cell or basophil and thereby sensitises the cell to the allergen. A subsequent exposure by the same allergen causes reactivation of the IgE, which then initiates the degranulation of the sensitised mast cell or basophil, probably in combination with co-stimulatory signals. The granules release histamine and other inflammatory chemical mediators, such as cytokines, interleukins, leukotrienes, and prostaglandins, into the surrounding tissue causing several systemic effects, such as vasodilation, mucous secretion, nerve stimulation and smooth muscle contraction. This results in the typical symptoms of acute allergic conditions, e.g. rhinorrhea, itchiness, dyspnea, and anaphylaxis. Depending on the individual, the particular allergen, and/or the route by which the allergen invades the organism, the symptoms can be system-wide or localised to particular organs or body regions. After the chemical mediators of the acute response subside, late phase responses may yet occur due to the migration of other leukocytes such as neutrophils, lymphocytes, eosinophils and macrophages to the initial site. The reaction is usually seen 4-6 hours after the original reaction and can last from 1-2 days. Cytokines from mast cells may also play a role in the persistence of long-term effects.

Allergic diseases may also be classified according to the predominant symptoms which they are associated with. For example, the invention comprises the treatment of patients suffering from seasonal allergic rhinitis (hay fever), perennial allergic rhinitis, allergic sinusitis, allergic conjunctivitis, and combinations thereof, such as rhinosinusitis; asthma, allergic bronchopulmonary aspergillosis, hypersensitivity pneumonitis; atopic dermatitis, urticaria; and any forms of food allergy, or house dust allergy, or an allergy to any of the allergen mentioned herein.

The scope of the invention extends over the treatment of any allergic patient without restriction, but also over the prophylactic treatment of any person without an allergy who is however at risk of developing an allergy. Such risk may be based on environmental factors such as a high level of exposure to common allergens, or on personal predisposition which may or may not be genetically determined, e.g. through polymorphisms of genes for the high-affinity IgE receptor β-chain, IL4, and CD14. In a further preferred embodiment, the method of the invention is used for the prophylactic or curative treatment of pediatric patients having a manifest allergic disease or an increased risk of developing an allergy. According to another embodiment, it is used for the prophylactic or curative treatment of parents of such pediatric patients.

While the invention is particularly suitable for the treatment of all allergic patients who are, according to commonly accepted criteria, candidates for a conventional hyposensitisation therapy (in particular subcutaneously administered hyposensitisation therapy), such as expressed in the WHO Position Paper, Allergen immunotherapy: therapeutic vaccines for allergic diseases (Geneva, Jan. 27-29, 1997), it offers the advantage that it is, due to its superior safety and tolerability, also suitable for many of those patients who have not been eligible for conventional hyposensitisation therapy because of the presence of certain risk factors or relative contraindications.

This is because the claimed method is potentially much safer than conventional hyposensitisation therapy. A major drawback of subcutaneous immunotherapy, i.e. the subcutaneous injection of allergens into an allergic patient, are allergic side effects. These side effects can be either local, at the site of injection. They are caused by the allergen crosslinking surface bound IgE on mast cells residing in the subcutis and the dermis, leading to mast cell degranulation and and subsequent allergic inflammation. The allergic side effects, however, can also be systemic. Systemic side effects are caused by allergen inadvertently injected into small subcutaneous blood vessels, or allergens diffusing into the subcutaneous blood vessels. From there the allergens may be transported to other organs such as the lung or distant sites of the skin, where they can again bind to surface IgE on mast cells and cause mast cell degranulation, resulting in asthma or hives. The most feared allergic side effect, however, is caused by allergens binding to surface IgE on basophil granulocytes in the blood. Degranulation of basophils leads to an allergic shock, a so called anaphylactic shock, that may result in death.

The epicutaneous administration of allergens according to the present invention is believed to be associated with none of the above side effects. Because the epidermis does not contain any mast cells there will be no local side effects. And because the epidermis does not contain any blood vessels, no allergen will reach the circulation, therefore avoiding systemic allergic side effects.

It is therefore a further embodiment of the invention which provides that patients are treated as described herein who exhibit either of the following risk factors: serious immunopathologic and immunodeficiency diseases, malignancy, severe psychological disorders, treatment with beta-blockers, ACE-inhibitors or AT-II antagonists; severe asthma which is uncontrolled by pharmacotherapy and/or irreversible airways obstruction; significant cardiovascular diseases which increase the risk of side-effects from epinephrine; children under 5 years of age.

A further advantage of the method of the invention is that it is much more convenient to the patient than conventional subcutaneous hyposensitisation therapy, and that it does not require injections which are considered painful or unpleasant by many patients. Therefore, its use is very promising even for patients who are poorly compliant with injectable therapies.

Yet a further advantage of the method of the invention is that it is potentially more cost-effective than conventional subcutaneous hyposensitisation therapy, as it is suitable for self-administration by the patients and does therefore not require frequent visits to a doctor's office.

The invention is further illustrated by means of the following example which should not be understood as intended to limit the scope of the invention.

### EXAMPLE

Adhesive patches containing a total of 100 micrograms of grass pollen extract (approx. 2 micrograms of each of the major grass pollen allergens) per patch were prepared as follows. The grass pollen extract, which had a specific activity in the range of between 5000 and 7000 protein nitrogen units (PNU/g) or 200 IR/g (biological units), was mixed with vaseline (pharmaceutical grade; 1.5 ml per patch). The mixture was then filled and sealed into polyethylene pouches having a size of 3.2×5 cm which comprised perforations on one side. The non-perforated side of each pouch was subsequently attached to a strip of commercially available medical adhesive tape of a size of 6×9 cm (Hydrofilm™, Paul Hartmann A G, Heidenheim, Germany). Each of the patches was covered with a strip of protective foil and sealed into paper pouches.

In a clinical study, 20 individuals suffering from hay fever were treated with the patches. Treatment was begun before the pollen season. For administration, a non-lesional skin area of the upper arm of each individual having the dimensions of approx. 4×4 cm was selected. The area was covered with a strip of household adhesive tape (Tesafilm®, Beiersdorf A G, Hamburg. Germany); subsequently, the adhesive tape was stripped off. The tape-stripping procedure was repeated 5 times. Thereafter, an adhesive patch comprising the grass pollen extract—as described above—was administered to the pre-treated skin area. The patch was removed after 48 hours. The pre-treatment and patch administration procedure was repeated in weekly intervals until each individual had received a total of 12 patches.

The severity of the pollen allergy was assessed by nasal provocation assays (NPT) with grass pollen extract before and after the treatment. The provocation tests were conducted according to an adapted method based on previously described nasal provocation testing, e.g. in: Reichelmann H, Bachert C, Goldschmidt O, Hauswald B, Klimek L, Schlenter W W, Tasman A J, Wagenmann M. Position statement. Allergo J (2002) 11:29-36.

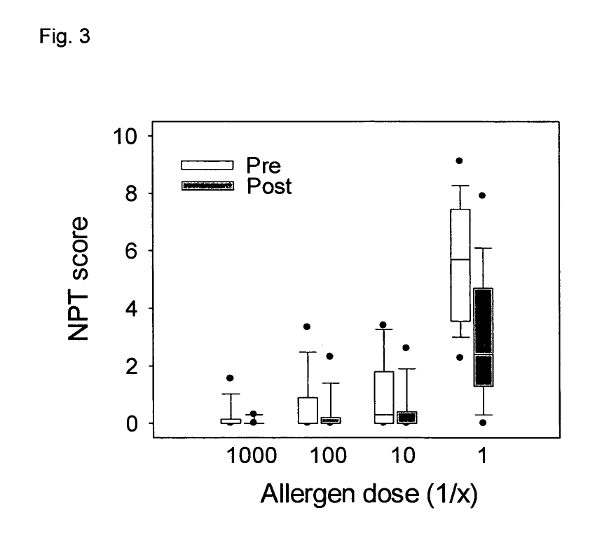

In short, the first step of the test was that the patient's symptom score was recorded prior to provocation, using a scale from 0 to 12. Then, using a nasal spray bottle, a placebo solution was sprayed into each nostril as a negative control, and symptom scores were recorded again. In the next step, a 1:1000 dilution of a grass pollen extract was sprayed into either nostril and, after 10 minutes, the patient's symptom score was evaluated again. Subsequently, a 1:100 dilution was administered, thereafter a 1:10 dilution, and finally the undiluted pollen allergen solution having an activity of approx. 100 RI were administered in the same manner, each time followed by a score evaluation. In result, it was observed that the NPT scores were improved substantially after treatment, as shown in FIG. 3. The x-coordinate of the diagram marks the dose or dilution of the allergen solution used for the test, ranging from 1:1000 on the left to 1 (undiluted) on the right. The y-axis represents the NPT score, i.e. the score for symptom severity. The boxes in the diagram represent the 25% and 75% percentiles, the lines in boxes show the mean values, the whiskers indicate the 5% and 95% percentiles, and the dots show outliers. Empty boxes and the associated lines, whiskers, and dots relate to the NPT scores before treatment, whereas the shaded boxes and their associated elements show the results after treatment. In summary, NPT scores were significantly improved (p<0.05) after the treatment, indicating successful hyposensitation.

1-25. (canceled)

26. A pharmaceutical composition for epicutaneous administration comprising an allergen and at least one excipient.

27. The composition of claim 26 wherein the allergen is selected from the group consisting of natural allergens, modified natural allergens, synthetic allergens, recombinant allergens, allergoids, and mixtures or combinations thereof.

28. The composition of claim 26 wherein the allergen is, or is obtained from, plant pollen, dust, animal dander, house dust mites, fungal spores, food, or the venom of ants, bees, or wasps, including any modifications, or recombinant or synthetic analogues thereof.

29. The composition of claim 26 wherein the allergen is a native protein or a fragment thereof, a recombinant protein, a fusion protein, a native peptide or a fragment thereof, a recombinant peptide, a chemical allergen, a synthetic compound mimicking an allergen, or a chemically or physically altered allergen.

30. The composition of claim 26 wherein the composition is formulated as an adhesive patch, an intradermal delivery device, a liquid, a gel, a spray, or a foam.

31. The composition of claim 30 wherein composition is formulated as an adhesive patch, and the adhesive patch exhibits a release area being adapted to release the allergen into the skin, and wherein the dimensions of the release area are in the range from about 0.5 to about 50 cm².

32. The composition of claim 30 wherein composition is formulated as an adhesive patch, and the adhesive patch comprises a substantially occlusive backing layer.

33. The composition of claim 30 wherein composition is formulated as an adhesive patch, and the adhesive patch comprises a pressure-sensitive adhesive layer, said layer being substantially hypoallergic.

34. The composition of claim 33 wherein at least a portion of the release area is not covered by the pressure-sensitive adhesive layer.

35. The composition of claim 30 comprising a reservoir compartment which is in liquid or semi-solid form, and wherein the allergen is incorporated within said reservoir compartment.

36. The composition of claim 30 wherein the content of the allergen per unit dose is in the range from about 0.1 to about 1,000 μg.

37. A pharmaceutical kit comprising the composition of claim 30 and printed instructions to perform a pre-treatment of a selected area of the skin and to subsequently administer the composition to the pre-treated area of the skin.

38. The kit of claim 37 wherein the pre-treatment effects the partial or complete dekeratinisation of the selected area of the skin.

39. The kit of claim 37 wherein the pre-treatment comprises tape-stripping.

40. The kit of claim 37 further comprising a means for pre-treating the selected area of the skin.

41. The kit of claim 40 wherein the means for pre-treating the selected area of the skin is an adhesive tape.

42. A method of treating a patient suffering from an allergy to an antigen comprising the steps of:
(a) pre-treating a selected area of the skin, and subsequently
(b) administering a pharmaceutical composition to the pre-treated area of the skin,
wherein the pharmaceutical composition comprises an antigen and at least one excipient, and wherein the antigen is selected from the group consisting of natural allergens, modified natural allergens, synthetic allergens, recombinant allergens, allergoids, and combinations thereof.

43. The method of claim 42 wherein the allergen is selected from the group consisting of natural allergens, modified natural allergens, synthetic allergens, recombinant allergens, allergoids, and mixtures or combinations thereof.

44. The method of claim 42 wherein the pre-treatment effects the partial or complete dekeratinisation of the selected area of the skin.

45. The method of claim 44 wherein the pre-treatment comprises tape-stripping.
